(19) United States
(12) Patent Application Publication
(10) Pub. No.: US 2003/0224028 A1
(43) Pub. Date: Dec. 4, 2003

(54) Metal complexes for promoting skin desquamation and/or stimulating epidermal renewal

(75) Inventor: Jean-Baptiste Galey, Aulnay-Sous-Bois (FR)

Correspondence Address: BURNS, DOANE, SWECKER & MATHIS, L.L.P., P.O. Box 1404, Alexandria, VA, 22313-1404, US

(73) Assignee: SOCIETE L'OREAL S.A., Paris (FR)

(21) Appl. No.: 10/435,030

(22) Filed: May 12, 2003

Related U.S. Application Data
(60) Provisional application No. 60/383,145, filed on May 28, 2002.

(30) Foreign Application Priority Data
May 13, 2002 (FR) 02/05855

(51) Int. Cl. A61K 7/00; A61K 31/69; A61K 31/555; A61K 31/295; A61K 31/28

(52) U.S. Cl. 424/401; 514/184; 514/64; 514/492; 514/502

(57) ABSTRACT

Certain Lₚ-Mⁿ⁺ metal complexes are well suited for promoting desquamation of the skin and/or stimulating epidermal renewal, for example preventing or treating dry skin and/or cutaneous signs of aging and/or pigmentation of the skin and/or acne-prone greasy skin; too, these are also suited for preventing/treating hyperkeratosis-induced skin disordes.

### CROSS-REFERENCE TO PRIORITY/PROVISIONAL APPLICATIONS

This application claims priority under 35 U.S.C. § 119 of FR-02/05855, filed May 13, 2002, and of provisional application Serial No. 60/383,145, filed May 28, 2002, both hereby expressly incorporated by reference. This application is also a continuation of said '145 provisional.

### BACKGROUND OF THE INVENTION

1. Technical Field of the Invention

The invention relates to the cosmetic use of at least one metal complex in a cosmetic composition comprising a physiologically acceptable medium, as an agent intended to promote desquamation of the skin and/or to stimulate epidermal renewal.

The metal complex according to the invention can thus be used for cosmetic purposes, for preventing or treating dry skin and/or cutaneous signs of aging and/or pigmentation of the skin and/or greasy skin with a tendency towards acne. As a variant, it can be used as a medicinal product, in particular for preparing a composition intended to prevent or treat skin disorders associated with hyperkeratosis.

2. Description of Related/Prior Art

Desquamation is a natural phenomenon associated with the fact that the epidermis, which constitutes the upper layer of the skin, is constantly regenerating.

The human epidermis consists of several strata of cells in which four types of cell are mainly found: keratinocytes, in great majority, melanocytes, Langerhans cells and Merkel cells. The distribution of these cells in several superimposed layers explains the stratified nature of the epidermis. The epidermis is conventionally divided into a basal layer of keratinocytes which constitutes the germinative layer of the epidermis, a “spiny” layer consisting of several layers of polyhedral cells arranged on the germinative cells, a “granular” layer consisting of flattened cells containing distinct cytoplasmic inclusions, keratohyalin granules, and, finally, an upper layer called horny layer (or stratum corneum), consisting of keratinocytes at the terminal stage of their differentiation, called corneocytes. Corneocytes are “mummified” anuclear cells derived from keratinocytes and are eliminated by desquamation. This surface loss is compensated for by the migration of cells from the basal stratum to the surface of the epidermis. This involves perpetual renewal of the epidermis. Forced elimination of the horny layer accelerates renewal and makes it possible to combat skin aging.

Corneocytes are mainly composed of a fibrous matrix containing cytokeratins, surrounded by a very resistant structure 15 nm thick, called horny or cornified envelope. The stacking of these corneocytes constitutes the horny layer which is responsible for the barrier function of the epidermis. During the normal process of desquamation, the uppermost corneocytes detach from the surface of the epidermis. Intercellular structures which derive from desmosomes, called corneosomes or corneodesmosomes, have been described in the horny layer. Recent studies have shown the major importance thereof in intercorneocyte cohesion and also in the desquamation process. Corneodesmosin, characterized moreover in the Applicant's application EP-A-0,972,042, is a protein of the horny layer of the epidermis, which is involved in intercorneocyte cohesion and which is a constituent of corneodesmosomes.

In the horny layer, a close correlation exists between cellular dissociation and proteolysis of certain corneodesmosomal components such as desmoglein I and corneodesmosin. Several serine proteases of the trypsin or chymotrypsin type appear to be involved in corneodesmosome proteolysis, such as, in particular, proteases of the chymotrypsin-like or trypsin-like type (Lundström A., Egelrud T., The Journal of Investigative Dermatology; 1988, 91:340-343 and 1990, 84:216-220).

Skin aging results from two distinct and independent processes which involve intrinsic or extrinsic factors.

Intrinsic or chronobiological aging corresponds to “normal” or physiological aging related to age. Extrinsic aging corresponds to aging caused, in general, by the environment, and more particularly to photo-aging due to exposure to the sun, to light or to any other radiation (EP-A2-0,815,840, Kligman, A. M. et al., Journal of Cutaneous Aging and Cosmetic Dermatology, Vol. 1, No. 1, pp. 5-12 (1988)).

The present invention relates in particular to intrinsic or physiological skin aging and also to extrinsic skin aging.

Skin aging generally results in the appearance of wrinkles and fine lines, in yellowing of the skin, which develops a wrinkled appearance, accompanied by the appearance of pigmentation marks, in disorganization of the elastin and collagen fibers, leading to a loss of elasticity, of suppleness and of firmness, or in the appearance of telangiectasia.

Changes in the skin due to intrinsic aging are the consequence of genetically programmed senescence involving endogenous factors. This intrinsic aging in particular results in a slowing down of the renewal of the epidermal cells, and the appearance of fine wrinkles or fine lines.

On the other hand, extrinsic aging results, in the dermis, in degradation of the collagen fibers, resulting in particular in clinical modifications such as thick wrinkles and the formation of a soft and weatherbeaten skin.

Some cosmetic agents promote desquamation, i.e., removal of the “dead” cells located at the surface of the horny layer of the epidermis. This is in particular the case of α-hydroxy acids (AHAs), such as lactic acid or glycolic acid, or β-hydroxy acids (BHAs), such as salicylic acid, which induce, by topical application at concentrations of a few %, visible desquamation after a few days. Their method of action is not known in detail, but is partly associated with the acidification that they induce in the various layers of the epidermis. Their desquamating effect is only observed if the pH of the products applied is less than 3. More precisely, the action of AHAs appears to be associated with the destruction of the protein body of the corneosome, which results in an acceleration of corneocyte separation.

Although lactic acid and salicylic acid accelerate corneocyte detachment in an abrupt and non-specific manner, 5-octanoylsalicylic acid appears to have a more targeted method of action, allowing a cleaner “cleavage” at the compactum/disjonctum interface, probably due to its lypophilic nature which allows it to penetrate via the layers of intercellular lipids.

In all cases, faced with this attack, the epidermis reacts immediately through a physiological response which results in a local irritation and in an acceleration of epidermal turnover in order to re-establish the thickness and the functions of the stratum corneum.

### SUMMARY OF THE INVENTION

The present invention thus provides novel prodesquamating compounds which do not have the irritant side effects of AHAs and of the other known keratolytic agents.

In addition, the desire to maintain a young appearance always leads to the incessant search for novel compounds and/or for novel compositions for maintaining or improving the appearance of the skin.

It has now surprisingly and unexpectedly been determined that certain metal complexes exhibiting hydrolytic properties find, in this respect, various applications in cosmetics and in dermatology.

Some metal complexes are already known for their hydrolytic properties (see, for example, Chin, Acc. Chem. Res. 1991, 24: 145). The use of metal complexes (ZnC₁₂ or CaC₁₂) of N,N′-dibenzylethylenediamine-N,N′-diacetic acid derivatives as depigmenting agents for human skin is also known from document EP-0,820,763 B1, as is a method of depigmenting and/or bleaching colored or marked skin.

On the other hand, it has never been described in the prior art that metal complexes constitute excellent selective and non-irritant cosmetic active agents capable of promoting desquamation and/or stimulating epidermal renewal and, consequently, finding applications in the treatment of cutaneous signs of skin aging, of dry skin, of hyperpigmentation, of greasy skin with a tendency towards acne and of all skin disorders engendered by hyperkeratosis.

The present invention therefore features the cosmetic use (regime or regimen) of at least one metal complex in a cosmetic composition comprising a physiologically acceptable medium, as an agent intended to promote desquamation of the skin and/or to stimulate epidermal renewal. Advantageously, said metal complex is a cosmetic agent which contributes to combating dry skin and/or cutaneous signs of aging and/or greasy skin with a tendency towards acne.

The metal complexes according to the invention can thus be used for cosmetic purposes, in a cosmetic composition, as a cosmetic agent for preventing or treating dry skin and/or cutaneous signs of aging and/or greasy skin with a tendency towards acne.

The invention therefore also features these cosmetic uses of the metal complexes. The present invention also features a method of cosmetic treatment of dry skin and/or of cutaneous signs of aging and/or of greasy skin with a tendency towards acne, comprising the topical application to the skin of a composition comprising, in a physiologically acceptable medium, at least one metal complex according to the invention.

The present invention also features a method of cosmetic treatment for decreasing pigmentation of the skin, comprising the topical application to the skin of a composition comprising, in a physiologically acceptable medium, at least one metal complex described in claim 5.

The invention also relates to a regime or regimen of cosmetic treatment intended to promote desquamation and/or stimulate epidermal renewal, characterized in that it comprises the topical application to the skin of a composition comprising, in a physiologically acceptable medium, a cosmetically acceptable amount of at least one metal complex according to the invention in order to promote said desquamation and/or stimulation. Advantageously, the method of cosmetic treatment is suitable for skin chosen from: dry skin, skin which exhibits cutaneous signs of aging, skin which exhibits hyperpigmentation or greasy skin with a tendency towards acne.

As a variant, the metal complex can be used as a medicinal product, in particular for preparing a pharmaceutical or dermatological composition comprising a physiologically acceptable medium, said composition being intended to prevent or treat skin disorders associated with hyperkeratosis, such as parapsoriasis, psoriasis, ichtyosis or lichen.

### DETAILED DESCRIPTION OF BEST MODE AND SPECIFIC/PREFERRED EMBODIMENTS OF THE INVENTION

The expression “physiologically acceptable medium” is understood to mean a medium compatible with the skin, the mucous membranes, the nails, the scalp and the hair.

The metal complexes according to the invention can optionally take different forms once solubilized in various solvents such as water, in particular dimerize as binuclear complexes or oligomerize as polynuclear complexes, while at the same time conserving their properties.

For the purpose of the present invention, the metal complexes are characterized by a specific hydrolytic activity; said hydrolytic activity is evaluated for its ability to accelerate at least 2-fold the hydrolysis, in aqueous solution at neutral or slightly alkaline pH, or in a water-acetonitrile or water-acetone mixture, of a substrate chosen from p-nitrophenol phosphate, p-nitrophenol acetate and p-nitroaniline acetate.

This method of evaluation is an adaptation of studies described, inter alia, by Kimura et al. (J. Am. Chem. Soc. 1990, 112: 5805).

This method is based on following, as a function of time, the hydrolysis of a model substrate: p-nitrophenol phosphate, p-nitrophenol acetate or p-nitroaniline acetate at neutral or slightly alkaline pH in aqueous solution or in a water-acetone or water-acetonitrile mixture advantageously comprising 10% to 50% of acetone or of acetonitrile, preferentially 20%, so as to allow solubilization of some complexes or substrates.

This method essentially consists in successively adding to a UV cuvette:

a buffer, advantageously a tris, phosphate or borate buffer, preferentially a tris buffer, at a concentration of between 10 and 100 mM, advantageously 50 mM, at a pH of between 7 and 9, advantageously at pH 8,

a solution of between 10⁻⁵ and 10⁻² M, advantageously 10 mM, of metal complex according to the invention in water or in a water-acetonitrile or water-acetone mixture, and

a solution of model substrate at a concentration of between 2 and 20 mM, advantageously at 10 mM, in acetonitrile or acetone.

The increase in optical density at 410 nm at a temperature of between 30° and 50° C., advantageously between 35° and 40° C., preferentially at 37° C. is then followed as a function of time for 1 hour.

The hydrolytic activity of the complex is determined by the ratio between the slope of the line obtained in the presence of complex and that of the line obtained in a control experiment without complex, which measures spontaneous hydrolysis of the substrate.

Thus, the metal complexes according to the present invention are those which accelerate at least 2-fold the hydrolysis of a substrate chosen from p-nitrophenol phosphate, p-nitrophenol acetate and p-nitroaniline acetate, at neutral or slightly alkaline pH, in aqueous solution or in a mixture consisting of water and acetonitrile or of water and acetone.

Advantageously, the metal complexes according to the invention correspond to formula (I) below:

Lₚ-Mⁿ⁺ (I)

in which,

M represents a metal chosen from zinc, cobalt, copper, iron, lanthanum, palladium, manganese, molybdenum, europium, cerium and zirconium;

n denotes a number chosen from 2, 3 and 4;

L represents a complexing agent chosen from a bidentate, a tridentate, a tetradentate, a pentadentate and a hexadentate;

p denotes a number chosen from 1, 2 and 3.

The invention also relates to the optical and/or geometric isomers of the metal complexes described above, alone or as a mixture in any proportions, and also to the physiologically acceptable salts of these derivatives.

The term “bidentate, tridentate, tetradentate, pentadentate or hexadentate complexing agent” is intended to mean the agents conventionally used to complex metal ions and having at least two donor groups which participate in the coordination of the metal ion, chosen from carboxylates, amines, carbonyls, alcohols, oximes, phenols, ethers, thiols, sulphonates and aromatic amines.

The classes of complexing agents preferred according to the present invention are chosen from:

macrocyclic polyamines, advantageously derivatives of 1,5,9-triazacyclododecane of formula (II), cyclen of formula (III) or the derivatives of 1,4,7-triazacyclononane of formula (IV)

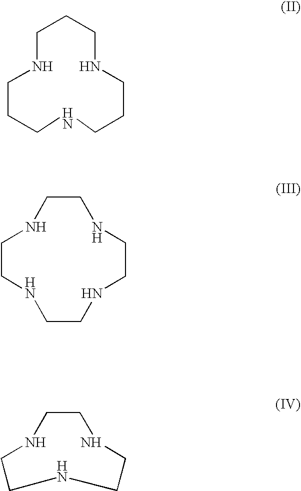

amino alcohols, advantageously N,N-bis(2-hydroxyethyl)ethylenediamine of formula (V), 2-(2-aminoethylamino)ethanol of formula (VI) or 1-(2-hydroxyethyl)piperazine of formula (VII)

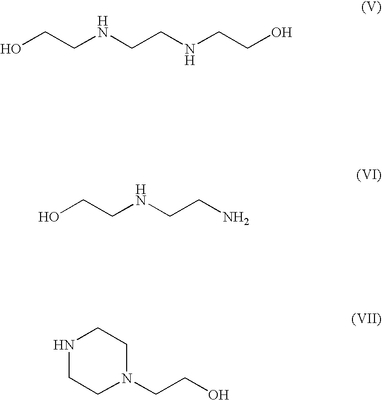

aminocarboxylic acids, advantageously histidine of formula (VIII) or N-(3,5-dimethoxybenzyl)-ethylenediamine-N,N′,N′-triacetic acid of formula (IX) described in patent application WO 94/11338;

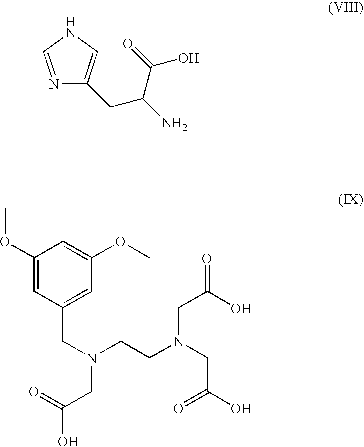

polyamines, advantageously trisaminoethylamine (tren) of formula (X) or N-(3,4,5-trimethoxybenzyl)-N′{2-[2-(3,4,5-trimethoxybenzylamino)ethylamino]ethyl}ethane-1,2-diamine of formula (XI) or N,N′,N″-tris(3,4,5-trimethoxybenzyl)triethylenetetramine of formula (XII) described in French patent application No. 96/07541;

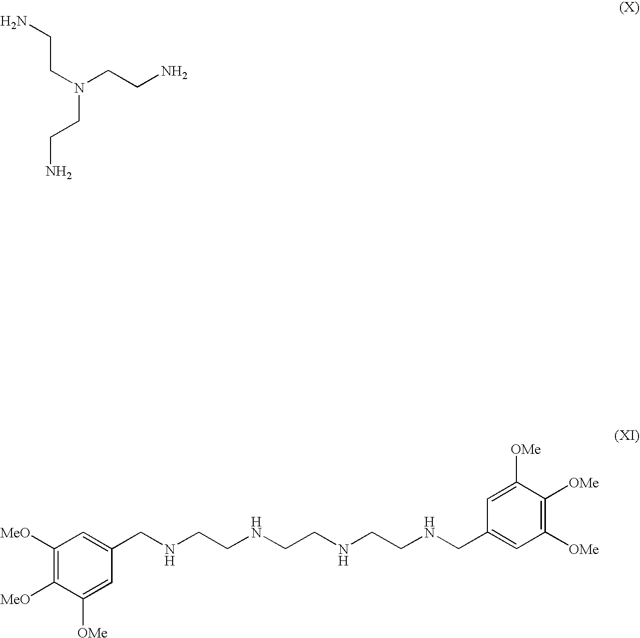

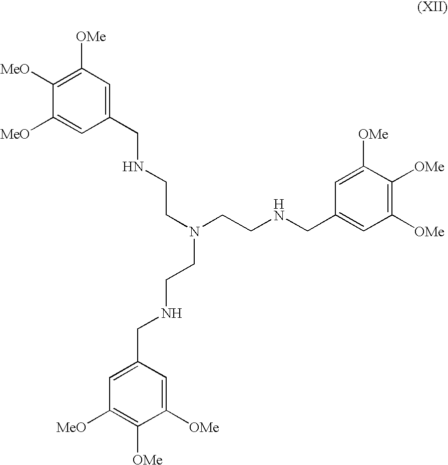

aromatic amines, advantageously 2,2′-dipyridylamine of formula (XIII), tris(2-benzimidazolylmethyl)amine of formula (XIV) and the derivatives of 5-methyl 3-tert-butyltris(pyrazolyl 1-borate) type of formula (XV),

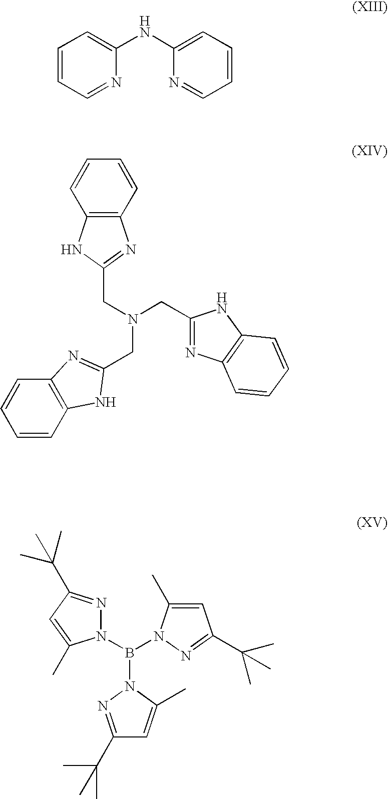

Of course, according to the invention, the metal complexes according to the invention can be used alone or as a mixture and in any proportion.

The amount of metal complex which can be used according to the invention depends, of course, on the desired effect and must be in an amount which is effective for promoting desquamation of the skin and/or stimulating epidermal renewal.

By way of example, the amount of metal complex according to the invention which can be used according to the invention can range, for example, from 0.01% to 50%, and preferably from 0.1% to 10%, of the total weight of the composition.

Advantageously, the metal complexes according to the invention are used in cosmetic (hair) compositions in combination with antidandruff active agents, desquamation of the scalp making it possible to strengthen the effectiveness of antidandruff agents and thus to make their action more effective.

Advantageously, the antidandruff active agents according to the invention are chosen from zinc pyrithione, piroctone olamine, selenium disulphide, climbazole, undecylenic acid, ketoconazole, ciclopirox, octopirox, and a complex formed by tropolone and a divalent metal salt, advantageously of zinc, of copper or of calcium.

The dermatological or pharmaceutical composition which can be used according to the invention can be ingested, injected or applied to the skin (to any skin area of the body), the hair, the nails or the mucous membranes (buccal, jugal, gingival, genital or conjunctival mucous membranes). Depending on the method of administration, the composition according to the invention can be in all the pharmaceutical forms normally used, particularly in cosmetology. A preferred composition of the invention is a cosmetic composition intended for topical application.

For topical application to the skin, the composition which can be used according to the invention can in particular be in the form of an aqueous or oily solution or of a dispersion of the lotion or serum type, of emulsions of liquid or semi-liquid consistency of the milk type, obtained by dispersion of a fatty phase in an aqueous phase (O/W) or vice versa (W/O), or of suspensions or emulsions of soft consistency of the cream or aqueous gel type or which are anhydrous, or else of microcapsules or microparticles, or of vesicular dispersions of the ionic and/or non-ionic type. These compositions are prepared according to the usual methods.

The composition which can be used according to the invention can also be a composition for hair care, and in particular a shampoo, a hair-setting lotion, a treating lotion, a styling cream or gel, a composition for dyeing (in particular oxidation dyeing) optionally in the form of dye shampoos, restructuring lotions for the hair, a permanent-wave composition (in particular a composition for the first stage of a permanent-wave operation), a composition for reducing and/or stabilizing natural hair loss in men, advantageously a lotion or a gel, an antiparasitic shampoo, etc.

The amounts of the various constituents of the compositions which can be used according to the invention are those conventionally used in the fields considered.

These compositions in particular constitute cleansing, protective, treatment or care creams for the face, for the hands, for the feet, for the large anatomical folds, or for the body (for example, day creams, night creams, make-up removing creams, foundation creams or antisun creams), liquid foundations, make-up removing milks, protective or care body milks, after-sun milks, lotions, gels or foams for skin care, such as cleansing lotions, antisun lotions or artificial tanning lotions, bath compositions, deodorizing compositions comprising a bactericidal agent, aftershave gels or lotions, depilatory creams, compositions for combating insect bites, pain-control compositions, or compositions for treating certain skin diseases, such as eczema, rosacea, psoriasis, lichens or severe pruritus.

The compositions which can be used according to the invention can also consist of solid preparations constituting cleansing bars or soaps.

The compositions which can be used according to the invention can also be packaged in the form of an aerosol composition also comprising a pressurized propellant.

In a known manner, the composition according to the invention can also contain the adjuvants which are usual in the cosmetics and dermatological fields, such as hydrophilic or lipophilic gelling agents, hydrophilic or lipophilic active agents, preserving agents, antioxidants, solvents, fragrances, fillers, screening agents, pigments, odor absorbers and dyestuffs. The amounts of these various adjuvants are those conventionally used in the field considered, and, for example, from 0.01% to 20% of the total weight of the composition. These adjuvants, depending on their nature, can be introduced into the fatty phase, into the aqueous phase and/or into the lipid spherules. In any event, these adjuvants, and also the proportions thereof, will be chosen so as not to harm the desired properties of the compound according to the invention.

When the composition of the invention is an emulsion, the proportion of the fatty phase may range from 5% to 80% by weight, and preferably from 5% to 50% by weight, relative to the total weight of the composition. The oils, the waxes, the emulsifiers and the co-emulsifiers used in the composition in the form of an emulsion are chosen from those conventionally used in the domain considered. The emulsifier and the co-emulsifier are generally present, in the composition, in a proportion ranging from 0.3% to 30% by weight, and preferably from 0.5% to 20% by weight, relative to the total weight of the composition. The emulsion may also contain lipid vesicles.

As oils which can be used in the composition of the invention, mention may, for example, be made of:

hydrocarbon oils of animal origin, such as perhydrosqualene;

hydrocarbon oils of plant origin, such as liquid triglycerides of fatty acids containing from 4 to 10 carbon atoms and the liquid fraction of karite butter;

synthetic esters and ethers, in particular of fatty acids, such as oils of formulae R¹COOR² and R¹OR² in which R¹ represents the residue of a fatty acid containing from 8 to 29 carbon atoms, and R² represents a branched or unbranched hydrocarbon chain containing from 3 to 30 carbon atoms, such as, for example, purcellin oil, isononyl isononanoate, isopropyl myristate, 2-hexylethyl palmitate, 2-octyldodecyl stearate, 2-octyldodecyl erucate, isostearyl isostearate; hydroxylated esters such as isostearyl lactate, octyl hydroxystearate, octyldodecyl hydroxystearate, diisostearyl malate, triisocetyl citrate, heptanoates, octanoates and decanoates of fatty alcohols; polyol esters such as propylene glycol dioctanoate, neopentyl glycol diheptanoate and diethylene glycol diisononanoate; and esters of pentaerythritol such as pentaerythrityl tetraisostearate;

linear or branched hydrocarbons of inorganic or synthetic origin, such as volatile or non-volatile paraffin oils and their derivatives, petroleum jelly, polydecenes, hydrogenated polyisobutene such as parleam oil;

fatty alcohols having from 8 to 26 carbon atoms, such as cetyl alcohol, stearyl alcohol and their mixture (cetylstearyl alcohol), octyldodecanol, 2-butyloctanol, 2-hexyldecanol, 2-undecylpentadecanol, oleyl alcohol or linoleyl alcohol;

partially hydrocarbon-based and/or silicon-based fluorinated oils such as those described in document JP-A-2-295912;

silicone oils such as volatile or non-volatile polymethylsiloxanes (PDMS) having a linear or cyclic silicone-containing chain, which are liquid or pasty at ambient temperature, in particular cyclopolydimethylsiloxanes (cyclomethicones) such as cyclohexasiloxane; polydimethylsiloxanes containing alkyl, alkoxy or phenyl groups, pendent or at the end of a silicon-containing chain, groups having from 2 to 24 carbon atoms; phenyl silicones such as phenyltrimethicones, phenyldimethicones, phenyltrimethylsiloxydiphenyl siloxanes, diphenyldimethicones, diphenylmethyldiphenyl trisiloxanes, 2-phenylethyltrimethyl siloxysilicates and polymethylphenyl siloxanes;

mixtures thereof.

As emulsifiers and co-emulsifiers which can be used in the invention, mention may, for example, be made of O/W emulsifiers such as fatty acid and polyethylene glycol esters, in particular PEG-100 stearate, and fatty acid and glycerine esters such as glyceryl stearate, and also W/O emulsifiers such as oxyethylenated poly(methylcetyl)(dimethyl)methyl-siloxane available under the commercial name ABIL WE09 from the company Degussa Goldschmidt.

As hydrophilic gelling agents, mention may in particular be made of carboxyvinyl polymers (carbomer), acrylic copolymers such as acrylate/alkyl acrylate copolymers, polyacrylamides, polysaccharides, natural gums and clays; and, as lipophilic gelling agents, mention may be made of modified clays such as bentones, metal salts of fatty acids, hydrophobic silica and polyethylenes.

As fillers which can be used in the composition of the invention, mention may, for example, be made, besides pigments, of silica powder; talc; starch crosslinked with octenylsuccinic anhydride marketed by the company National Starch under the name DRY FLO PLUS (28-1160); polyamide particles and in particular those sold under the name ORGASOL by the company Atochem; polyethylene powders; microspheres based on acrylic copolymers, such as those made of ethylene glycol dimethacrylate/lauryl methacrylate copolymer sold by the company Dow Corning under the name POLYTRAP; expanded powders such as hollow microspheres and in particular the microspheres marketed under the name EXPANCEL by the company Kemanord Plast or under the name MICROPEARL F 80 ED by the company Matsumoto; silicone resin microbeads such as those marketed under the name TOSPEARL by the company Toshiba Silicone; and mixtures thereof. These fillers may be present in amounts ranging from 0% to 20% by weight, and preferably from 1% to 10% by weight, relative to the total weight of the composition or of the preparation according to the invention.

It is also possible to introduce into the composition according to the invention UVA and/or UVB screening agents chosen from organic screening agents and inorganic screening agents, optionally coated to make them hydrophobic.

According to another aspect, a subject of the invention is a composition comprising at least the combination of at least one metal complex and at least one other agent chosen from desquamating agents other than said metal complex, moisturizers, depigmenting or propigmenting agents; antiglycation agents; NO-synthase inhibitors; agents for reducing and/or stabilizing natural hair loss; agents which act on dermal or epidermal macromolecules and/or which prevent their degradation; agents which stimulate the proliferation of fibroblasts and/or of keratinocytes or which stimulate the differentiation of keratinocytes; muscle relaxants; antimicrobial agents; tensioning agents; antipollution agents and/or free-radical scavengers; calmants; active agents which are lipolytic or which have a favorable, direct or indirect, activity on decreasing adipose tissue; agents which act on the microcirculation; agents which act on the energy metabolism of cells; and mixtures thereof.

1. Desquamating Agents and Moisturizers:

The term “desquamating agent” is intended to mean any compound capable of acting:

either directly on desquamation by promoting exfoliation, such as β-hydroxy acids, in particular salicylic acid and its derivatives (including 5-n-octanoylsalicylic acid); α-hydroxy acids, such as glycolic acid, citric acid, lactic acid, tartaric acid, malic acid or mandelic acid; urea; gentisic acid; oligofucoses; cinnamic acid; extract of Saphora japonica; resveratrol;

or on the enzymes involved in desquamation or the degradation of corneodesmosomes, glycosidases, stratum corneum chymotryptic enzyme (SCCE), or even other proteases (trypsin, chymotrypsin-like). Mention may also be made of aminosulphonic compounds and in particular (N-2-hydroxyethylpiperazine-N-2-ethane)sulphonic acid (HEPES); derivatives of 2-oxothiazolidine-4-carboxylic acid (procysteine); derivatives of alpha-amino acids of the glycine type (as described in EP-0,852,949); honey; sugar derivatives such as O-octanoyl-6-D-maltose and N-acetylglucosamine.

The term “moisturizer” is intended to mean:

either a compound which acts on the barrier function, for the purpose of maintaining the moisturization of the stratum corneum, or an occlusive compound. Mention may be made of ceramides, sphingoid-based compounds, lecithins, glycosphingolipids, phospholipids, cholesterol and its derivatives, phytosterols (stigmasterol, β-sitosterol or campesterol), essential fatty acids, 1,2-diacylglycerol, 4-chromanone, pentacyclic triterpenes such as ursolic acid, petroleum jelly and lanolin;

or a compound which directly increases the water content of the stratum corneum, such as threalose and its derivatives, hyaluronic acid and its derivatives, glycerol, pentanediol, sodium pidolate, serine, xylitol, sodium lactate, polyglyceryl acrylate, ectoin and its derivatives, chitosan, oligosaccharides and polysaccharides, cyclic carbonates, N-lauroylpyrrolidonecarboxylic acid and N-α-benzoyl-L-arginine;

or a compound which activates sebaceous glands, such as steroidal derivatives (including DHEA) and vitamin D and its derivatives.

These compounds may represent from 0.001% to 30%, and preferably from 0.01% to 20%, of the total weight of the composition according to the invention.

The composition according to the present invention comprising the moisturizing agents mentioned above is advantageously intended for the prevention or treatment of drying out of the skin, and in particular of xerosis.

2. Depigmenting or Propigmenting Agent:

The depigmenting agents that may be incorporated into the composition according to the present invention comprise, for example, the following compounds: cojic acid; ellagic acid; arbutin and its derivatives such as those described in applications EP-895,779 and EP-524,109; hydroquinone; aminophenol derivatives such as those described in applications WO 99/10318 and WO 99/32077, and in particular N-cholesteryloxycarbonyl-para-aminophenol and N-ethyloxycarbonyl-para-aminophenol; iminophenol derivatives, in particular those described in application WO 99/22707; L-2-oxothiazolidine-4-carboxylic acid or procysteine, and also its salts and esters; ascorbic acid and its derivatives, in particular ascorbyl glucoside; and plant extracts, in particular extracts of liquorice, of mulberry and of skullcap, without this list being limiting.

As propigmenting agents, mention may be made of the extract of burnet (Sanguisorba officinalis) marketed by the company MARUZEN and extracts of chrysanthemum (Chrysanthemum morifolium).

The composition according to the present invention comprising the depigmenting agents mentioned above is advantageously intended for the prevention or treatment of hyperpigmentations, in particular pigmentation marks associated with skin aging.

As regards the composition containing the propigmenting agents mentioned above, it is preferably intended to treat hair turning grey.

3. Anti-Glycation Agent:

The term “anti-glycation agent” is intended to mean a compound which prevents and/or decreases the glycation of skin proteins, in particular of dermal proteins such as collagen.

Examples of anti-glycation agents are plant extracts from the Ericacea family, such as an extract of blueberry (Vaccinium angusfifollium); ergothioneine and its derivatives; hydroxystilbenes and their derivatives, such as resveratrol and 3,3′,5,5′-tetrahydroxystilbene. These anti-glycation agents are described in applications FR 99/16166, FR 00/08158, FR 99/09267 and FR 99/16168, respectively. Resveratrol is particularly preferred for use in this invention.

The composition according to the invention comprising an anti-glycation agent as defined above can advantageously be used to prevent or treat signs of skin aging, in particular to prevent or treat loss of tonicity and/or of elasticity of the skin.

4. NO-Synthase Inhibitor:

Examples of NO-synthase inhibitors which are suitable for use in the present invention comprise in particular an extract of a plant of the species Vitis vinifera “which is in particular marketed by the company Euromed under the name Leucocyanidines de raisins extra, or by the company Indena under the name Leucoselect®, or finally by the company Hansen under the name Extrait de marc de raisin; an extract of a plant of the species Olea europaea which is preferably obtained from olive tree leaves and is in particular marketed by the company VINYALS in the form of a dry extract, or by the company Biologia & Technologia under the commercial name Eurol BT; and an extract of a plant of the species Gingko biloba which is preferably a dry aqueous extract of this plant sold by the company Beaufour under the commercial name Ginkgo biloba extrait standard.

The composition according to the invention comprising an NO-synthase inhibitor as defined above may advantageously be used to prevent or treat signs of skin aging and/or sensitive skin.

5. Agents for Reducing and/or Stabilizing Natural Hair Loss:

retinoids, and in particular retinol;

sulphur and sulphur-containing derivatives;

zinc salts such as zinc lactate, gluconate, pidolate, carboxylate, salicylate and/or cysteate;

selenium chloride;

vitamin B6 or pyridoxine;

the mixture of capryloylglycine, sarcosine and extract of cinnamomum zeylanicum marketed in particular by the company SEPPIC under the commercial name Sepicontrol A5®;

an extract of Laminaria saccharina marketed in particular by the company SECMA under the commercial name Phlorogine®;

an extract of Spiraea ulmaria marketed in particular by the company SILAB under the commercial name Sebonormine®;

extracts of plants of the species Arnica montana, Cinchona succirubra, Eugenia caryophyllata, Humulus lupulus, Hypericum perforatum, Mentha piperita, Rosmarinus officinalis, Salvia oficinalis and Thymus vulgaris, all marketed, for example, by the company MARUZEN;

an extract of Serenoa repens marketed in particular by the company EUROMED;

extracts of plants of the genus Silybum;

plant extracts containing sapogenins, and in particular the diosgenin-rich or hecogenin-rich Dioscorea extracts; and

extracts of Eugenia caryophyllata containing eugenol and eugenyl glucoside;

b 2,4-diaminopyrimidine 3-oxide or 2,4-DPO described in patent application WO 96/09048;

2,4-diamino-6-piperidinopyrimidine 3-oxide or “Minoxidil” described in U.S. Pat. Nos. 4,139,619 and 4,596,812.

These compounds are, for example, present in the composition according to the invention in the region of 0.001% to 10% by weight, and better still in the region of 0.01% to 5% by weight, relative to the total weight of the composition.

6. Agent Which Acts on Dermal or Epidermal Macromolecules and/or Which Prevents Their Degradation:

Among the active agents which stimulate dermal macromolecules, mention may be made of those which act:

either on collagen synthesis, such as extracts of Centella asiatica; asiaticosides and derivatives; ascorbic acid or vitamin C and its derivatives; synthetic peptides such as lamin, biopeptide CL or palmitoyloligopeptide marketed by the company SEDERMA; peptides extracted from plants, such as the soybean hydrolysate marketed by the company COLETICA under the commercial name Phytokine®; and plant hormones such as auxins;

or on elastin synthesis, such as the extract of Saccharomyces Cerivisiae marketed by the company LSN under the commercial name Cytovitin®; and the extract of the alga Macrocystis pyrifera marketed by the company SECMA under the commercial name Kelpadelie®;

or on glycosaminoglycan synthesis, such as the product of milk fermentation by lactobacillus vulgaris, marketed by the company BROOKS under the commercial name Biomin yogourth®; the extract of the brown alga Padina pavonica marketed by the company ALBAN MÜLLER under the conunercial name HSP3®; and the extract of Saccharomyces cerevisiae available in particular from the company SILAB under the commercial name Firmalift® or from the company LSN under the commercial name Cytovitin®;

or on fibronectin synthesis, such as the extract of the zooplankton Salina marketed by the company SEPORGA under the commercial name GP4G®; the yeast extract available in particular from the company ALBAN MÜLLER under the commercial name Drieline®; and the palmitoyl pentapeptide marketed by the company SEDERMA under the commercial name Matrixil®;

or on metalloprotease (MMP) inhibition, such as, more particularly, MMP 1, 2, 3 or 9. Mention may be made of: retinoids and derivatives, isoflavonoids, oligopeptides and lipopeptides, lipoamino acids, the malt extract marketed by the company COLETICA under the commercial name Collalift®; extracts of blueberry or of rosemary; lycopene; isoflavones, their derivatives or the plant extracts containing them, in particular extracts of soybean (marketed, for example, by the company ICHIMARU PHARCOS under the commercial name Flavosterone SB®), of red clover, of flax, of kakkon, or of sage;

or on the inhibition of serine proteases such as leukocyte elastase or cathepsin G. Mention may be made of: the peptide extract of Leguminosa seeds (Pisum sativum) marketed by the company LSN under the commercial name Parelastyl®; heparinoids; and pseudodipeptides.

Among the active agents which stimulate epidermal macromolecules, such as fillagrin and keratins, mention may in particular be made of the extract of lupin marketed by the company SILAB under the commercial name Structurine®; the extract of Fagus sylvatica beech buds marketed by the company GATTEFOSSE under the commercial name Gatuline®; and the extract of the zooplankton Salina marketed by the company SEPORGA under the commercial name GP4G®.

The composition according to the invention containing one or more of the compounds above is particularly suitable for use in preventing or treating cutaneous signs of aging, in particular loss of firmness and/or elasticity of the skin.

7. Agent Which Modulates the Proliferation of Fibroblasts or Keratinocytes and/or the Differentiation of Keratinocytes:

The agents which stimulate the proliferation of fibroblasts which can be used in the composition according to the invention may, for example, be chosen from plant proteins or polypeptides, extracted in particular from soybean (for example a soybean extract marketed by the company LSN under the name Eleseryl SH-VEG 8® or marketed by the company SILAB under the commercial name Raffermine®); and plant hormones such as giberrellins and cytokinins.

The agents which stimulate the proliferation of keratinocytes, which can be used in the composition according to the invention, in particular comprise retinoids such as retinol and its esters, including retinyl palmitate; extracts of nut cakes marketed by the company GATTEFOSSE; and extracts of Solanum tuberosum marketed by the company SEDERMA.

The agents which stimulate the differentiation of keratinocytes comprise, for example, minerals such as calcium; the extract of lupin marketed by the company SILAB under the commercial name Photopreventine®; sodium beta-sitosteryl sulphate marketed by the company SEPORGA under the commercial name Phytocohesine®; and the extract of corn marketed by the company SOLABIA under the commercial name Phytovityl®.

The composition according to the invention comprising these compounds is preferentially intended to be used for preventing or treating cutaneous signs of aging.

8. Muscle Relaxant:

The muscle relaxants which can be used in the composition according to the invention comprise calcium inhibitors such as alverine and its salts, chloride channel openers such as diazepam, and inhibitors of catecholamines and of acetylcholine, such as the hexapeptide argireline R marketed by the company LIPOTEC.

The composition according to the invention comprising these compounds is preferentially intended to be used for preventing and treating cutaneous signs of aging, and in particular wrinkles.

9. Antimicrobial Agent:

The antimicrobial agents which may be used in the composition according to the invention may in particular be chosen from 2,4,4′-trichloro-2′-hydroxydiphenyl ether (or triclosan), 3,4,4′-trichlorobanilide, phenoxyethanol, phenoxypropanol, phenoxyisopropanol, hexamidine isethionate, metronidazole and its salts, miconazole and its salts, itraconazole, terconazole, econazole, ketoconazole, saperconazole, fluconazole, clotrimazole, butoconazole, oxiconazole, sulfaconazole, sulconazole, terbinafine, ciclopirox, ciclopiroxolamine, undecylenic acid and its salts, benzoyl peroxide, 3-hydroxybenzoic acid, 4-hydroxybenzoic acid, phytic acid, N-acetyl-L-cysteine acid, lipoic acid, azelaic acid and its salts, arachidonic acid, resorcinol, 2,4,4′-trichloro-2′-hydroxydiphenyl ether, 3,4,4′-trichlorocarbanalide, octopirox, octoxyglycerin, octanoylglycine, caprylyl glycol, 10-hydroxy-2-decanoic acid, dichlorophenylimidazole dioxolane and its derivatives described in WO 93/18743, farnesol, phytosphingosines and their mixtures.

The preferred antimicrobial agents are triclosan, phenoxyethanol, octoxyglycerin, octanoylglycine, 10-hydroxy-2-decanoic acid, caprylyl glycol, farnesol and azelaic acid.

By way of example, the antimicrobial agent may be used in the composition according to the invention in an amount representing from 0.1% to 20%, and preferably from 0.1% to 10%, of the total weight of the composition.

The composition containing the antimicrobial agent is particularly suitable for use in the treatment of greasy skin with a tendency towards acne, acne, or dandruff of the scalp.

10. Tensioning Agent:

The term “tensioning agent” is intended to mean a compound capable of exerting tension on the skin, the effect of which is to temporarily fade out irregularities on the skin's surface, such as wrinkles and fine lines.

Among the tensioning agents which can be used in the composition according to the present invention, mention may in particular be made of:

(1) polyurethane latices or acrylic-silicone latices, in particular those described in EP-1,038,519, such as a propylthio(polymethyl acrylate), propylthio(polymethyl methacrylate) and propylthio(polymethacrylic acid) grafted polydimethylsiloxane, or alternatively a propylthio(polyisobutyl methacrylate) and propylthio(polymethacrylic acid) grafted polydimethylsiloxane. Such grafted silicone polymers are in particular sold by the company 3M under the commercial names VS 80, VS 70 or LO 21,

(2) soybean or wheat plant proteins, and/or

(3) sodium and magnesium silicates (laponites).

The compositions according to the invention comprising the tensioning agents above are advantageously intended for the treatment of cutaneous signs of aging, in particular wrinkles and fine lines.

11. Antipollution Agent or Free-Radical Scavenger:

The term “antipollution agent” is intended to mean any compound capable of trapping ozone, monocyclic or polycyclic aromatic compounds such as benzopyrene and/or heavy metals such as cobalt, mercury, cadmium and/or nickel. The term “free-radical scavenger” is intended to mean any compound capable of trapping free radicals.

As ozone-trapping agents which can be used in the composition according to the invention, mention may in particular be made of vitamin C and its derivatives, including ascorbyl glucoside; phenols and polyphenols, in particular tannins, ellagic acid and tannic acid; epigallocatechin and natural extracts containing it; extracts of olive tree leaf; extracts of tea, in particular of green tea; anthocyans; extracts of rosemary; phenol acids, in particular chlorogenic acid; stilbenes, in particular resveratrol; sulphur-containing amino acid derivatives, in particular S-carboxymethylcysteine; ergothioneine; N-acetylcysteine; carotenoids such as crocetin; and various raw materials such as the mixture of arginine, histidine ribonucleate, mannitol, adenosine triphosphate, pyridoxine, phenylalanine, tyrosine, and hydrolysed RNA, marketed by Laboratoires Sérobiologiques under the commercial name CPP LS 2633-12F®, the water-soluble fraction of corn marketed by the company SOLABIA under the commercial name Phytovityl®, the mixture of extract of fumetory and of extract of lemon marketed under the name Unicotrozon C-49® by the company Induchem, and the mixture of extracts of ginseng, of apple, of peach, of wheat and of barley, sold by the company PROVITAL under the commercial name Pronalen Bioprotect®.

As agents which trap monocyclic or polycyclic aromatic compounds, which can be used in the composition according to the invention, mention may in particular be made of tannins such as ellagic acid; indole derivatives, in particular indole-3-carbinol; extracts of tea, in particular of green tea, extracts of water hyacinth or eichornia crassipes; and the water-soluble fraction of corn marketed by the company SOLABIA under the commercial name Phytovityl®.

The free-radical scavengers which can be used in the composition according to the invention comprise, besides certain antipollution agents mentioned above, vitamin E and its derivatives such as tocopheryl acetate; bioflavonoids; coenzyme Q10 or ubiquinone; certain enzymes such as catalase, superoxide dismutase, lactoperoxidase, glutathione peroxydase and quinone reductases; glutathione; benzylidene camphor; benzylcyclanones; substituted naphthalenones; pidolates; phytanetriol; gamma-oryzanol; lignans; and melatonin.

12. Calmants:

As calmants which can be used in the composition according to the invention, mention may be made of the raw materials which are effective in inhibiting at least one of the enzymes chosen from phospholipases, lipooxygenases and human prostaglandin synthetases, among which: pentacyclic triterpenes and the extracts of plants (for example Glycyrrhiza glabra) containing them such as β-glycyrrhetinic acid and its salts and/or its derivatives (glycyrrhetic acid monoglucuronide, stearyl glycyrrhetinate, 3-stearoyloxyglycyrrhetic acid), ursolic acid and its salts, oleanolic acid and its salts, betulinic acid and its salts, extract of Paeonia suffruticosa and/or lactiflora, calophyllum oil, the salts of salicylic acid and in particular zinc salicylate, phycosaccharides (hydrolysed algin or hydrolysed algin and zinc sulphate) from the company Codif, phlorogine (laminaria saccharina) from Secma, canola oil, tamanu oil, calophyllum oil, β-bisabolol and camomile extracts, allantoin, Sepivital EPC (phosphoric diester of vitamin E and C) from Seppic, omega-3 unsaturated oils such as musk rose oils, blackcurrant seed oils, echium oils or fish oils, omega plankton (plankton extract) from Secma, lipacid C8G (capriloyl glycine) from Seppic, Seppicalm VG (sodium palmitoylproline and nymphea alba) from Seppic, extract of rosebay willow-herb, extract of pygeum, Soothex (extract of boswellia serrata) from Quest, phytoplenolin (extract of centipeda cunnighami) from Bio-Botanica, Helioxine (extract of helianthus annuus) from Silab, Sensiline (linum usitatissimum) from Silab, tocotrienols, extract of Cola nitida, piperonal, extract of clove, extract of rosebay willow-herb (Epilobium Angustifolium), aloe vera, bacocalmine (extract of bacopa moniera) from Séderma, phytosterols, cortisone, hydrocortisone, indomethacin and betamethasone.

The amount of the compounds which are inhibitors of PLA2, Lox, PGHS-1 depends of course on the desired effect and can therefore vary within a wide range.

To give an order of magnitude, it can be used in an amount representing from 0.001% to 10% of the total weight of the composition, and preferentially in an amount representing from 0.01% to 5% of the total weight of the composition.

13. Active Agents Which are Lipolytic or Which Have a Favorable, Direct or Indirect, Activity on Decreasing Adipose Tissue:

Among the derivatives which may promote lipolysis, the following may be found:

1) phosphodiesterase inhibitors such as:

xanthine derivatives, such as caffeine and its derivatives, in particular the 1-hydroxyalkylxanthines described in FR-A-2,617,401, caffeine citrate, theophylline and its derivatives, theobromine, acefylline, aminophylline, chloroethyltheophylline, diprofylline, diniprophylline, etamiphylline and its derivatives, etofylline and proxyphylline;

combinations containing xanthine derivatives, such as the combination of caffeine and silanol (caffeine methylsilanetriol derivative), and for example the product marketed by the company Exsymol under the name cafeisilane C;

compounds of natural origin containing xanthine bases, and in particular caffeine, such as extracts of tea, of coffee, of guarana, of maté, of cola (Cola nitida) and in particular the dry extract of guarana fruit (Paulina sorbilia) containing 8% to 10% of caffeine;

ephedrine and its derivatives which may be found especially in natural form in plants such as Ma Huang (ephedra plant);

2) Plant extracts and extracts of marine origin, which are either active on the receptors to be inhibited, such as β-2-blockers or NPY-blockers (described in EP 838217), or inhibit the synthesis of LDL or VLDL receptors, or are active in stimulating β-receptors and G proteins, leading to the activation of adenyl cyclase. As plant extracts of this type, mention may, for example, be made of:

Garcinia cambogia,

extracts of Bupleurum chinensis,

extracts of climbing ivy (Hedera helix), of arnica (Arnica montana L), of rosemary (Rosmarinus officinalis N), of marigold (Calendula officinalis), of sage (Salvia officinalis L), of ginseng (Panax ginseng), of St. John's wort (Hypericum perforatum), of butcher's broom (Ruscus aculeatus L), of meadowsweet (Filipendula ulmaria L), of orthosiphon (Orthosiphon stamincus Benth), of birch (Betula alba), of pumpwood and of argan tree,

extracts of ginkgo biloba,

extracts of horsetail,

extracts of escin,

extracts of cangzhu,

extracts of Chrysanthemum indicum,

extracts of dioscorea plants rich in diosgenin or pure diosgenin or hecogenin, and derivatives thereof,

extracts of plants of the genus Armeniacea, Atractylodis platicodon, Sinommenum, Pharbitidis, Flemingia,

extracts of Coleus such as C. Forskohlii, C. blumei, C. esquirolii, C. scutellaroides, C. xanthantus and C. Barbatus, such as the extract of root of Coleus barbatus containing 60% of forskolin,

extracts of Ballote,

extracts of Guioa, of Davallia, of Terminalia, of Barringtonia, of Trema or of antirobia.

As extracts of marine origin, mention may be made of extracts of algae or of phytoplankton, such as rhodysterol or the extract of Laminaria digitata marketed under the name PHYCO R 75 by the company Secma, the alga skeletonema described in FR-2,782,921 or the diatoms described in FR-2,774,292.

3) Peptides or Proteins

the peptides derived from parathyroid hormone as described in FR-2,788,058 and FR-2,781,231 from Sederma or the peptides described in document FR-2,786,693, or even any other peptide having lipolytic properties,

protamines and derivatives thereof such as those described in document FR-A-2,758,724.

The amount of lipolytic active agent(s) may vary within a wide range and depends on the nature of the active agent(s) used. In general, the slimming active agent(s) is (are) present at a concentration ranging from 0.001% to 20%, and preferably from 0.1% to 10%, by weight relative to the total weight of the composition.

14. Agents Which Act on the Microcirculation:

The active agents which act on the microcirculation (vasoprotectors of vasodilators) are found in particular among flavonoids, ruscogenins, esculosides, escin extracted from common horsechestnut, nicotinates, heperidine methyl chalcone, ruscus, essential oils of lavender or of rosemary, and extracts of Ammi visnaga.

The amount of these active agents can vary within a wide range. In general, these active agents are present at a concentration ranging from 0.01% to 15%, and preferably from 0.05% to 10%, by weight relative to the total weight of the composition.

15. Agents Which Act on the Energy Metabolism of Cells:

This expression is intended to mean active agents which act on the energy metabolism of the skin, such as, for example, and in a nonlimiting manner, ATP synthesis, and also those involved in the respiratory chain of the cell or in the energy stores. In this respect, mention may be made of coenzyme Q10 (ubiquinone), cytochrome C, creatine or phosphocreatine.

As indicated above, the composition according to the invention can also contain UVA and/or UVB screening agents, in the form of organic or inorganic compounds, the latter optionally being coated to make them hydrophobic.

The organic screening agents may in particular be chosen from: anthranilates, in particular menthyl anthranilate; benzophenones, in particular benzophenone-1, benzophenone-3, benzophenone-5, benzophenone-6, benzophenone-8, benzophenone-9, benzophenone-12 and, preferentially, benzophenone-2 (oxybenzone) or benzophenone-4 (Uvinul MS40 available from BASF); benzylidenecamphors, in particular 3-benzylidenecamphor, benzylidenecamphosulphonic acid, camphor benzalkonium methosulphate, polyacrylamidomethylbenzylidene camphor, terephthalylidenedicamphorsulphonic acid, and preferentially 4-methylbenzylidenecamphor (Eusolex 6300 available from Merck); benzimidazoles, in particular benzimidazilate (Neo Heliopan AP available from Haarmann and Reimer), or phenylbenzimidazolesulphonic acid (Eusolex 232 available from Merck); benzotriazoles, in particular drometrizole trisiloxane, or methylenebisbenzotriazolyltetramethylbutylphenol (Tinosorb M available from Ciba); cinnamates, in particular cinoxate, DEA methoxycinnamate, diisopropyl methylcinnamate, glyceryl ethylhexanoate dimethoxycinnamate, isopropyl methoxycinnamate, isoamyl cinnamate, and preferentially ethocrylene (Uvinul N35 available from BASF), octyl methoxycinnamate (Parsol MCX available from Hoffmann La Roche) or octocrylene (Uvinul 539 available from BASF); dibenzoylmethanes, in particular butylmethoxydibenzoylmethane (Parsol 1789); imidazolines, in particular ethylhexyl dimethoxybenzylidene dioxoimidazoline; PABAs, in particular ethyl dihydroxypropyl PABA, ethylhexyldimethyl PABA, glyceryl PABA, PABA, PEG-25 PABA, and preferentially diethylhexylbutamidotriazone (Uvasorb HEB available from 3V Sigma), ethylhexyltriazone (Uvinul T150 available from BASF) or ethyl PABA (benzocaine); salicylates, in particular dipropylene glycol salicylate, ethylhexyl salicylate, homosalate or TEA salicylate; triazines, in particular anisotriazine (Tinosorb S available from Ciba); drometrizole trisiloxane.

The inorganic screening agents preferably consist of zinc oxide and/or titanium dioxide, preferably of nanometric size, optionally coated with alumina and/or stearic acid.

In order to further illustrate the present invention and the advantages thereof, the following specific examples are given, it being understood that same are intended only as illustrative and in nowise limitative.

### EXAMPLE 1

Example of Metal Complexes:

Synthesis of the N,N′-bis(dibenzyl)ethylene-diamine-N,N′-diacetic acid zinc complex 0.61 g of zinc acetate dihydrate are solubilized in 5 ml of water.

1 g of N,N′-bis(dibenzyl)ethylenediamine-N,N′-diacetic acid (L1, prepared as described in WO 94/11338) in solution in 10 ml of 1% aqueous ammonia is added.

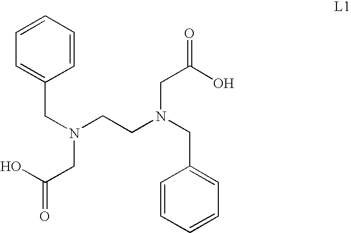

The mixture is stirred for 1 hour at ambient temperature.

The white precipitate is filtered through sintered glass and washed with water then dried under vacuum over P²O⁵.

0.86 g of white powder corresponding to the complex Zn-L1 is obtained.

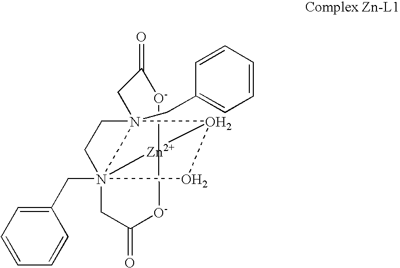

Elemental Analysis

Elemental analysis

| | C | H | N | O | Zn |
|---|---|---|---|---|---|
| Expected | 52.60 | 5.70 | 6.10 | 21.00 | 14.50 |
| Found | 53.35 | 5.59 | 5.71 | 21.87 | 13.30 |

Alternatively, the complexes according to the invention can be prepared extemporaneously by mixing a molar equivalent of metal salt with one to two molar equivalents of ligand in solution in an appropriate solvent.

Synthesis of L4 Ligand Cobalt Complex.

Thus, a 1 mM solution of N,N-bis(2-hydroxyethyl)-ethylenediamine (L2) zinc complex is prepared by mixing 3.7 mg of zinc perchlorate Zn(ClO₄)₂ (hexahydrate) and 3.0 mg of L2 ligand in 10 ml of water, and then stirring for 30 min at ambient temperature.

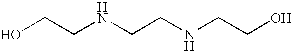

Similarly, a 1 mM solution of tris(2-benzimidazolylmethyl)amine (L3) zinc complex is prepared by mixing a molar equivalent of zinc perchlorate Zn(ClO₄)₂ (hexahydrate) and of L3 ligand in 10 ml of a 1:1 water:acetonitrile mixture, and then stirring for 30 min at ambient temperature.

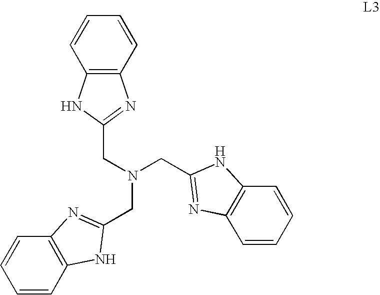

Finally, according to the same method, starting with cobalt chloride and with N-(3,4,5-trimethoxybenzyl)-N,N′-bis[2-(3,4,5-trimethoxybenzylamino)ethyl]ethane-1,2-diamine (L4, prepared according to French patent application No. 96/07541), a solution of L4 ligand cobalt complex is obtained.

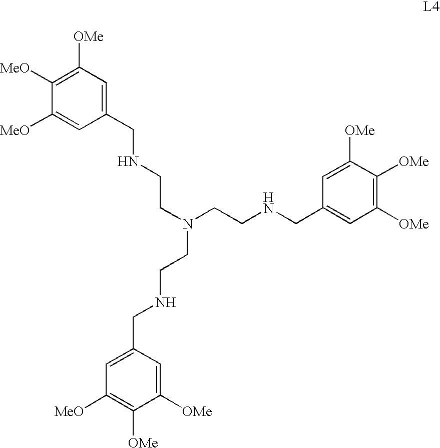

### EXAMPLE 2

Implementation of the Hydrolytic Properties of the Complex Co-L4:

The following are successively added to a UV cuvette:

2.6 ml of 50 mM tris buffer at pH 8,

300 μl of a 10 mM solution of complex Co-L4 in water, and

100 μl of a 10 mM solution of p-nitrophenol acetate in acetonitrile.

The increase in the optical density at 410 nm is then followed at 37° C. as a function of time for 1 hour using a visible UV spectrophotometer.

A ratio of 2:1 is observed between the slope of the line obtained and that of the line obtained in a control experiment without complex, which measures the spontaneous hydrolysis of the substrate.

### EXAMPLE 3

Compositions:

The following compositions are prepared in a manner which is conventional for those skilled in the art.

Prodesquamating cream for the face:

| | | |
|---|---|---|
| Complex Zn-L1 | | 2.00% |
| Sodium stearate | | 3.00% |
| Liquid petroleum jelly | | 6.00% |
| Alkyl paraben | | 0.05% |
| Potassium sorbate | | 10.00% |
| Stearyl alcohol | | 1.00% |
| Fragrance | | 1.00% |
| Water | qs | 100.00% |

Prodesquamating cream for the body:

| | | |
|---|---|---|
| Complex Co-L2 | | 5.0% |
| Jojoba oil | | 13.0% |
| Sipol wax | | 6.0% |
| Isopropyl palmitate | | 2.0% |
| Glycerol | | 15.0% |
| Alkyl paraben | | 0.5% |
| Fragrance | | 1.0% |
| Water | qs | 100.0% |

Prodesquamating care cream:

| | | |
|---|---|---|
| Complex Zn-L2 | | 1% |
| Oxyethylenated polyethylene glycol 50 | | 3% |
| Diglyceryl monostearate | | 3% |
| Liquid petroleum jelly | | 24% |
| Cetyl alcohol | | 5% |
| Water | qs | 100% |

Desquamating care cream for the body:

| | | |
|---|---|---|
| Complex Cu-L1 | | 0.5% |
| Sipol wax | | 6.0% |
| Glyceryl monostearate | | 1.5% |
| Sodium stearate | | 0.8% |
| Liquid petroleum jelly | | 6.0% |
| Isopropyl palmitate | | 2.0% |
| Glycerol | | 15.0% |
| Fragrance | | 0.3% |
| Water | qs | 100.0% |

Prodesquamating care cream:

| | | |
|---|---|---|
| Complex Zr-L1 | | 0.50% |
| Jojoba oil | | 13.00% |
| Alkyl paraben | | 0.05% |
| Potassium sorbate | | 0.30% |
| Cyclopentadimethylsiloxane | | 10.00% |
| Stearyl alcohol | | 1.00% |
| Stearic acid | | 4.00% |
| Polyethylene glycol stearate | | 3.00% |
| Vitamin E | | 1.00% |
| Glycerol | | 3.00% |
| Water | qs | 100.00% |

Each patent, patent application and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A regime or regimen for promoting desquamation of the skin and/or stimulating epidermal renewal, comprising administering to an individual in need of such treatment, a thus effective amount of at least one metal complex able to at least 2-fold accelerate the hydrolysis, in aqueous solution at neutral or slightly alkaline pH or in a water/acetonitrile or water/acetone mixture, of a p-nitrophenol phosphate, p-nitrophenol acetate or p-nitroaniline acetate substrate.

2. The regime or regimen as defined by claim 1, said at least one metal complex having the following formula (I):
Lp-Mⁿ⁺ (I)
in which, M is a metal selected from the group consisting of zinc, cobalt, copper, iron, lanthanum, palladium, manganese, molybdenum, europium, cerium and zirconium; n is a number selected from 2, 3 and 4; L is a complexing agent which comprises a bidentate, a tridentate, a tetradentate, a pentadentate or a hexadentate; p is a number selected from 1, 2 and 3.

3. The regime or regimen as defined by claim 2, said at least one complexing agent having at least two donor groups which participate in the coordination of the metal ion, selected from the group consisting of carboxylates, amines, carbonyls, alcohols, oximes, phenols, ethers, thiols, sulfonates and/or aromatic amines.

4. The regime or regimen as defined by claim 3, said at least one complexing agent being selected from the group consisting of macrocyclic polyamines, amino alcohols, aminocarboxylic acids, polyamines and/or aromatic amines.

5. The regime or regimen as defined by claim 2, said at least one complexing agent being selected from the group consisting of a derivative of 1,5,9-triazacyclododecane of formula (II), a cyclen of formula (III) or a derivative of 1,4,7-triazacyclononane of formula (IV): 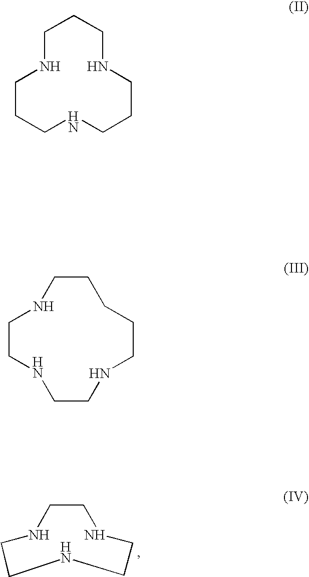
an N,N-bis(2-hydroxyethyl)ethylenediamine of formula (V), 2-(2-aminoethylamino)ethanol of formula (VI) or 1-(2-hydroxyethyl)piperazine of formula (VII): 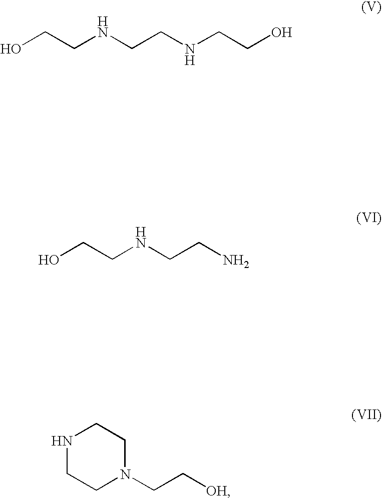
a histidine of formula (VIII) or N-(3,5-dimethoxybenzyl)ethylenediamine-N,N′,N′-triacetic acid of formula (IX): 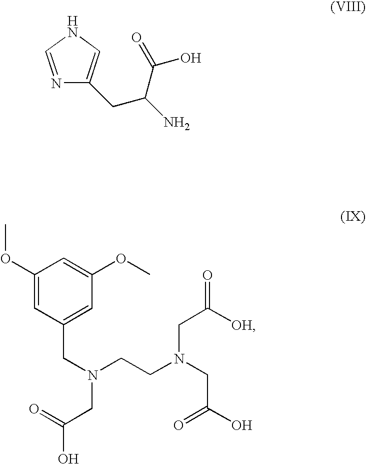
a trisaminoethylamine (tren) of formula (X) or N-(3,4,5-trimethoxybenzyl)-N′{2-[2-(3,4,5-trimethoxybenzylamino)ethylamino]ethyl}ethane-1,2-diamine of formula (XI) or N,N′,N″-tris(3,4,5-trimethoxybenzyl)triethylenetetramine of formula (XII): 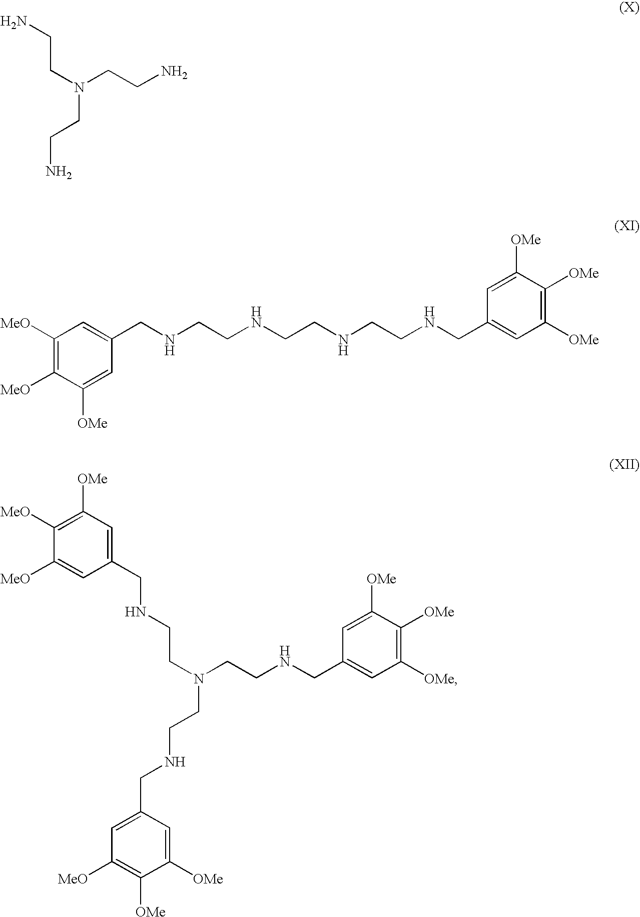
and/or 2,2′-dipyridylamine of formula (XIII), tris(2-benzimidazolylmethyl)amine of formula (XIV) or a derivative of 5-methyl 3-tert-butyltris(pyrazolyl 1-borate) type of formula (XV):

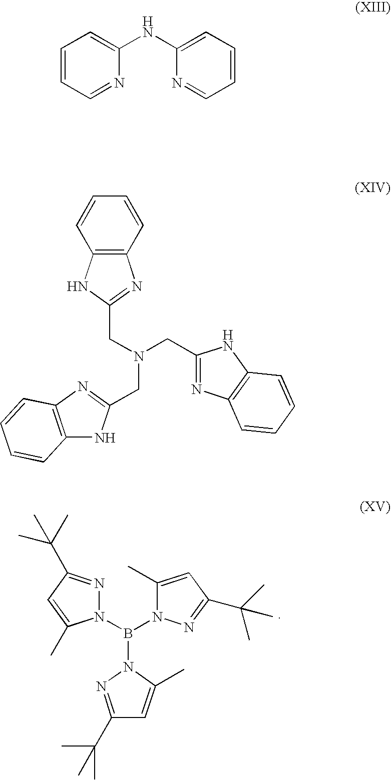

6. A regime or regimen for preventing or treating dry skin and/or cutaneous signs of aging and/or skin pigmentation and/or treating acne-prone greasy skin, comprising administering to an individual in need of such treatment, a thus effective amount of at least one metal complex able to at least 2-fold accelerate the hydrolysis, in aqueous solution at neutral or slightly alkaline pH or in a water/acetonitrile or water/acetone mixture, of a p-nitrophenol phosphate, p-nitrophenol acetate or p-nitroaniline acetate substrate.

7. The regime or regime as defined by claim 1, including the treatment of dandruff, comprising coadministering an effective amount of at least one antidandruff active agent.

8. The regime or regimen as defined by claim 7, said at least one antidandruff agent comprising zinc pyrithione, piroctone olamine, selenium disulfide, climbazole, undecylenic acid, ketoconazole, ciclopirox, octopirox, and/or a complex formed or tropolone and a divalent metal salt.

9. The regime or regimen as defined by claim 6, comprising topically applying onto the skin of such individual in need of such treatment, a thus effective amount of at least one metal complex able to at least 2-fold accelerate the hydrolysis, in aqueous solution at neutral or slightly alkaline pH or in a water/acetonitrile or water/acetone mixture, of a p-nitrophenol phosphate, p-nitrophenol acetate or p-nitroaniline acetate substrate.

10. A regime or regimen for decreasing skin pigmentation, comprising topically applying onto the skin of such individual in need of such treatment, a thus effective amount of at least one metal complex able to at least 2-fold accelerate the hydrolysis, in aqueous solution at neutral or slightly alkaline pH or in a water/acetonitrile or water/acetone mixture, of a p-nitrophenol phosphate, p-nitrophenol acetate or p-nitroaniline acetate substrate.

11. A regime or regimen for preventing or treating a hyperkeratosis-induced skin disorder, comprising administering to an individual in need of such treatment, a thus effective amount of at least one metal complex able to at least 2-fold accelerate the hydrolysis, in aqueous solution at neutral or slightly alkaline pH or in a water/acetonitrile or water/acetone mixture, of a p-nitrophenol phosphate, p-nitrophenol acetate or p-nitroaniline acetate substrate.

12. The regime or regimen as defined by claim 11, said hyperkeratosis-induced skin disorder comprising parapsoriasis, psoriasis, ichthyosis and/or lichen.

13. A cosmetic/dermatological composition comprising at least one metal complex as defined by claim 1 and at least one desquamating agent other than said metal complex, moisturizer in an amount representing from 0.001% to 30% of the total weight of the composition; depigmenting or propigmenting agent; anti-glycation agent; NO-synthase inhibitor; agent for reducing and/or stabilizing natural hair loss; agent which acts on dermal or epidermal macromolecules and/or which prevents degradation thereof; agent which stimulates the proliferation of fibroblasts and/or of keratinocytes or which stimulates the differentiation of keratinocytes; muscle relaxant; antimicrobial agent in an amount representing from 0. 1% to 20% of the total weight of the composition; tensioning agent; antipollution agent and/or free-radical scavenger; calmant; active agent which is lipolytic or which elicits activity on decreasing adipose tissue; agent which affects microcirculation; agent which affects energy metabolism of cells; and mixtures thereof.
